(19) United States
(12) Patent Application Publication
(10) Pub. No.: US 2004/0009473 A1
(43) Pub. Date: Jan. 15, 2004

(54) Kit and process for microbiological for on-site examination of a liquid sample

(76) Inventor: Christopher Pease, Littleton, MA (US)

Correspondence Address: MILLIPORE CORPORATION, 290 CONCORD ROAD, BILLERICA, MA, 01821, US

(21) Appl. No.: 10/457,631

(22) Filed: Jun. 9, 2003

Related U.S. Application Data
(60) Provisional application No. 60/387,253, filed on Jun. 7, 2002.

(51) Int. Cl. C12Q 1/70; C12Q 1/68; C12Q 1/66; C12M 1/34

(52) U.S. Cl. 435/5; 435/6; 435/8; 435/287.2

(57) ABSTRACT

A kit and process are provided for rapidly measuring, on-site, the microbe concentration in a liquid sample. The kit includes a filtration device that includes a filter on to which microbes are captured from the liquid stream. A first reagent is applied to lyse the microbial cells is used and then a second reagent is applied (may be simultaneously) to provide a means for detecting the presence of such cells, such as by luciferine and luciferase, radioactive tags or one or more PNA tags. The signal from the reaction of the second reagent with the lysed cells is then detected by an appropriately selected sensor such as a luminometer.

The invention relates to a kit and a process for effecting rapid microbiological examination of a liquid, on-site.

At the present time there is a need to test apparatus utilized in the preparation of materials such as food, beverages, cosmetics for microbial levels. It is current practice to utilize the apparatus to prepare a desired product, shut it down, clean-in-place and then test it for residual microbial levels before initiating the next process batch. The apparatus is first cleaned with an appropriate cleaning agent such detergents, hot water, caustic, enzymes, then finished by one or several rinses with cold rinse water. In one current process, a bleed stream of the rinse water is taken from the processing apparatus, and processed through a filter that retains the microbe. The microbes collected on the membrane then are cultured and measured. After a suitable microbial level is determined, that information is typically retroactively used for trend analysis. Generally, the membrane filtration method used for the microbial measuring process is conducted in a laboratory environment remote from the processing area. Unfortunately the process skill requirements, facility requirements and time-to result are time consuming and the food processing apparatus is used before the test results are available.

Accordingly, it would be desirable to provide a process for testing a materials processing apparatus such as a food, beverage or cosmetic or any other processing apparatus for microbial content that does not require a step of growing microbes. In addition, it would be desirable to provide such a process that can be conducted at the site of the processing apparatus. Such a process would provide a quick and convenient means for accurately measuring microbial levels in the apparatus.

### SUMMARY OF THE INVENTION

The present invention provides a kit and process for testing materials processing apparatus such as food processing apparatus for microbial contact prior to processing the material through the apparatus. The kit comprises a device and sampling port for removing a liquid without the requirement of a vacuum pump, e.g., sterile water from the material processing apparatus and for filtering the removed liquid through a filter that retains microbes. The volume of filtered liquid is measured so that the number of microbes per volume of filtered liquid can be subsequently determined. The sensitivity of the assay can be adjusted by modifying the filtered volume. The volume of filtered liquid is measured downstream from the filter so that the device is suitable for an extremely large variation in sample volume (from milliliters to a cubic meter, if no filter plugging occurs). Subsequent to filtration, a reagent capable of lysing cells and for reacting with the lysed cell components to produce a measurable radiant energy such as luminescent light is added to the device to contact and react with the microbes isolated on the filter. Subsequent to reaction, the device is positioned within a radiant energy measuring apparatus in order to measure the radiant energy emitted from the lysed cells. This measurement then is compared to a previously developed standard that provides a measure of radiant energy as a function of microbe concentration in order to determine microbe concentration in the sample. When the measurement comprises an acceptably low measurement, use of the material processing apparatus is resumed. An exemplary reagent comprises a mixture of luciferine and luciferase and an exemplary radiant energy measuring device is a luminometer.

### BRIEF DESCRIPTION OF THE DRAWINGS

FIG. 1 is an elevational view of a device useful in the present invention.

FIG. 1 is a sectional elevational view of this device of FIG. 1.

FIGS. 3 and 4 are similar views to FIGS. 1 and 2 but showing, respectively, only the intake body and the drainage body.

FIG. 5 is an enlargement of the part of FIG. 2 positioned at the bottom right.

FIG. 6 is a partial sectional view of a seal of the device of FIG. 1.

FIG. 7 is a sectional elevational view showing how the sampling device utilized in the invention is used for sampling the liquid to be examined.

FIG. 8 is a partial sectional view illustrating the use of this invention.

### DESCRIPTION OF SPECIFIC EMBODIMENTS

The present invention provides a kit and process for determining microbe concentration in a sample. The kit comprises a device for collecting and retaining a microbe sample as well as a reagent composition which lyses the cells of the collected microbes and which reacts with the lysed cells to produce an energy signal, such as luminescence. The kit is utilized with a device that can detect and measure radiant energy from a liquid sample such as a luminometer.

A suitable device for collecting and retaining a microbe sample and for reacting the microbe sample with a liquid reagent composition is the MicroPreSure™ filtration device available from Millipore Corporation of Billerica, Mass. USA and is described in International Application NO. PCT/IBOO/01908 and published as International Publication No. WO 01/48142 A1. It is to be understood that any device suitable for collecting a microbe by filtration from a liquid sample and which permits subsequent reaction with a liquid sample wherein the degree of reaction can be measured is suitable for the present invention. Generally, such devices include a filter supported on a porous substrate, a housing for the filter, a fluid inlet into the housing and a fluid outlet from the housing.

The reagent composition of the kit of this invention lyses the collected microbe cells and reacts with one or more components of the lysed cell to produce radiant energy that may be detected either visually or by machine. Suitable reagent compositions for lysing the cells include but are not limited to detergents, alcohols, esters, ethers and halogenated derivatives of methane, ethane, methylene and ethylene, as well as acetonitrile, and trimethylamine and other such known lysing agents. Methanol and ethanol are two examples of such lysing agents useful in the present invention. Luminescent-inducing reagents, include but are not limited to luciferine and luciferase that react with the ADP and/or ATP of the lysed cell to create a luminescent signal (see JP 7213297A); or radioactive tags and peptide nucleic acid (PNA) tags (See U.S. Pat. No. 5,773,571 and WO 02/27036A2) that react with the DNA or RNA of the microbes to provide a radiant signal that can be detected and other known radiant energy detection systems for indicating the presence and/or type of microbe present can be used in the present invention. Other reagents such as pyruvate orthophosphate dikinase (PPDK) that converts existing cell AMP to ATP which is then detected by the reaction with luciferine/luciferase (see GB2317892A), adenylate kinase that converts cellular ADP to ATP that is then detected by the luminescent reagent (See GB2304892) or reagents which reduce the background noise of the system, such as ATP hydrolase (See U.S. Pat. No. 5,908,751) or adenosine phosphate deaminase (See U.S. Pat. No. 5,891,702) to delete any non-microbial ATP from the solution or the filter before lysing of the cells so that only the cellular ATP signal is detected, can also be used in this system.

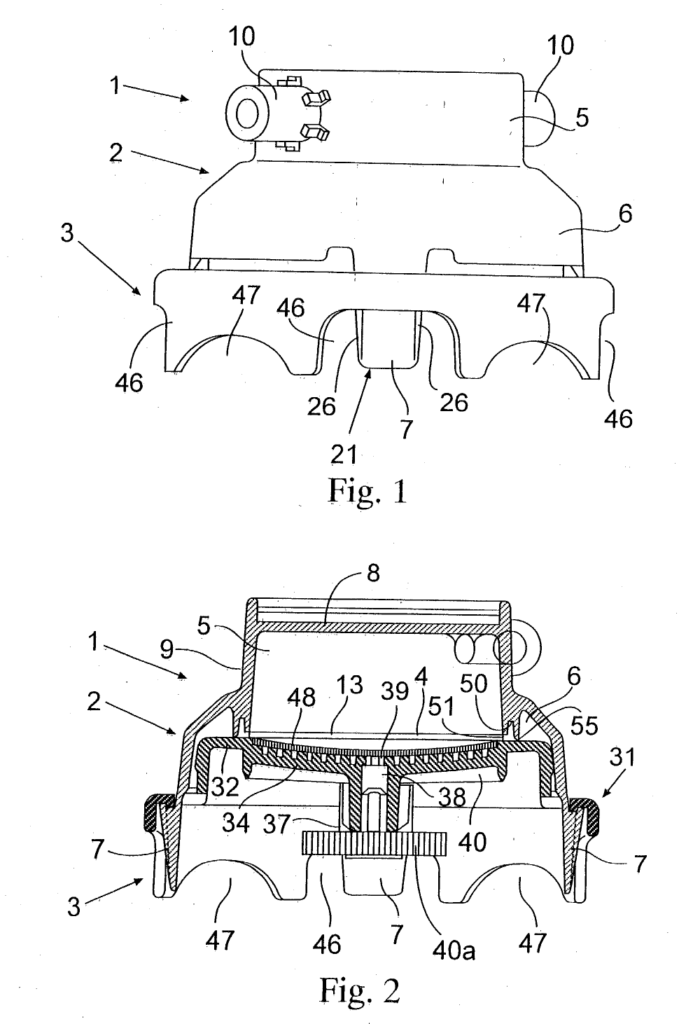

The device 1 for microbiological examination of a sample of liquid under pressure shown in the drawings, and notably in FIGS. 1 and 2 has in general terms a symmetry of revolution around a central axis. The device 1 includes an intake body 2, a drainage body 3 and a filtering membrane 4.

The intake body 2 has a reservoir 5, a skirt 6 which is connected externally to the reservoir 5 and four latching tabs 7 which extend projecting from the skirt 6, in an axial direction.

The reservoir 5 has a transparent end wall 8 and a lateral wall 9.

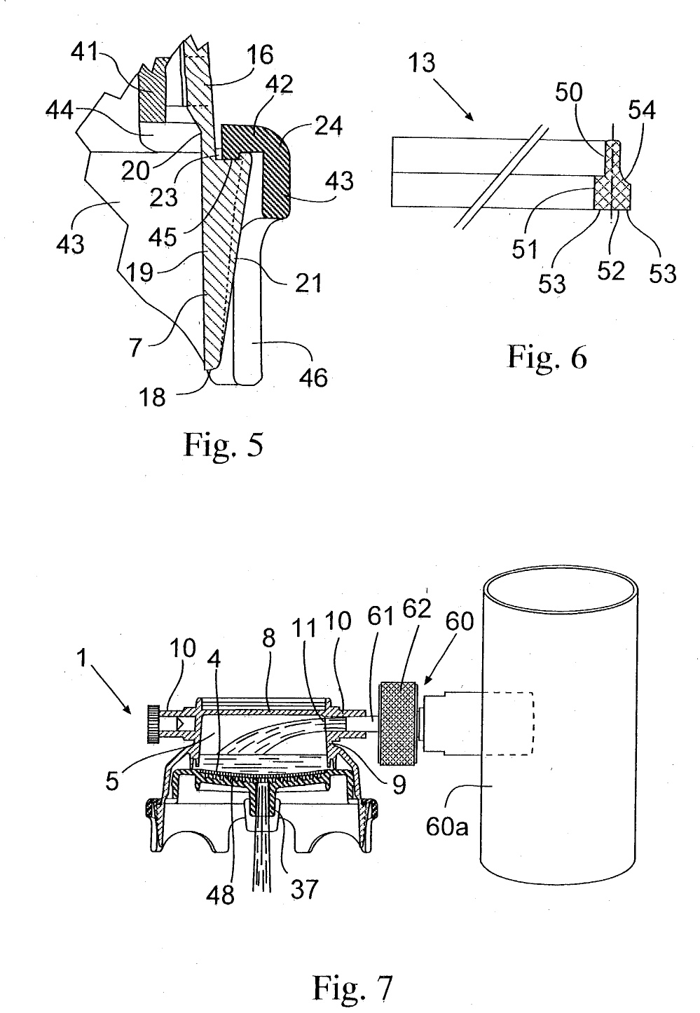

One diametrically conduit 10 extend projecting outward from the lateral wall 9, above the skirt 6, constituting a female luer connector adapted to receive internally a male luer connector, as will be explained below with the reference to FIG. 5, the passage internal to the conduit 10 being continued by an aperture 11 made in the wall 9, this aperture being in immediate proximity to the end wall 8.

The lateral wall 9 finishes at the end opposite the end wall 8 in an edge forming part of a seal 13, a groove 14 being made to that effect in the rigid part of the wall 9, as will be explained in more detail subsequently with the help of FIGS. 2 and 3 and 6.

The skirt 6 is connected to reservoir 5 by the outside of the lateral wall 9, at a level situation between the groove 14 and the conduits 10. The skirt 6 has a truncated-cone shaped wall 15 and a cylindrical wall 16, the skirt 6 being connected to the wall 9 by the small-diameter end of the wall 15. The connection between the walls 15 and 16 is made by the large-diameter end of the wall 15.

Each of the latching tabs 7 has a general outline in the form of a trapezium symmetrical with respect to the axial direction, the side forming the free end 18 of the tab 7 being parallel to the one by which this tab is connected to the skirt 6, and more precisely to the edge of the wall 16, the tab 7 narrowing gradually between its connection to the skirt 6 and its free end.

On either side of each tab 7, a notch 17 is made in the wall 16, over a certain distance from the edge thereof.

Each tab 7 has, from its free end 18, an internal surface 19 which is straight, that is to say parallel to the axial direction, as far as a dihedral 20 from which the surface 19 is inclined inward and towards the wall 16.

The external surface 21 of each tab 16 is inclined outward and towards the wall 16. The surface 21 extends between the surface 18 and a transversely oriented surface 22 which connects the surface 21 and a groove 23 positioned between an external shoulder 24 whose surface 22 constitutes the edge and a surface 25 offset inward with respect to the surface 21. The surface 25 comprises the continuation of the external surface of the wall 16.

As can be seen better in FIG. 4, the drainage body 3 has a circular table 30 and a skirt 31 disposed in a step around the table 30.

The latter has an annular transverse wall 32 delimited on the opposite side from the skirt 31 by a surface 44 which is flat in the main but having a slight bevel towards the outside.

The internal periphery of the wall 32 is connected to a wall 34 delimited, on the side of the surface 33, by a surface 35 which is concave in the main, offset with respect to the surface 32 in the axial direction, towards the skirt 31, the perimeter of the surface 35 and the internal periphery of the surface 33 being connected by a slightly truncated-cone shaped surface 36.

The wall 34 is connected centrally to a conduit 37 whose internal passage is extended into the wall 34 by an output aperture 38. Concentric drainage channels 38 are positioned into the wall 34 from the surface 35. Radially oriented channels (not visible in the drawings) also are made, with the same depth as the channels 39, these radial channels opening of course into the output aperture 38, through which flows the liquid drained by the channels made in the wall 34 hollowed out with respect to the surface 35.

An annular rib 40 is positioned at the junction between the walls 32 and 34. The rib 40 projects with respect to the walls 32 and 34 on the side of the skirt 31. The rib tapers towards its free end in a V-shaped profile, so that this end constitutes a sharp edge.

The table 30 also has a tubular lateral wall 41 that is connected by one end to the wall 32 and the opposing end is connected to the skirt 31. The latter has a transversely oriented annular wall 42 and an axially oriented cylindrical wall 43, the wall 42 being connected by one of its ends to the wall 41 and by the other to the wall 43.

In the wall 42, in proximity to the wall 41, four openings 44 are made, which have between them the same angular spacing as between the latching tabs 7, that is to say they are spaced out from one another by 90°. The openings 44 have an outline corresponding to the largest outline of the tabs 7, so that the latter can each pass through a respective opening 44.

Each opening 44 is bordered on the external side by an axially oriented tooth 45 projecting on the opposite side from the table 30. Each tooth 45 extends projecting over a height corresponding to the depth of the groove 23 and has a thickness less than the width of the groove 23, the distance separating each tooth 45 from the wall 43 being greater than the thickness of the shoulder 24 (See FIG. 5).

At the level of each opening 44, the wall 43 has a notch 48 of general rectangular form with rounded corners, extending over approximately two thirds of the height of the wall 43 and over a width that is approximately twice the width of the latching tabs 7.

The wall 43 also has four notches 47, each disposed halfway between two successive notches 46, the notches 47 have a rounded form whose maximum height corresponds approximately to one third of the height of the wall 43.

The drainage body 3 also has a porous pad 48 (not depicted in FIG. 4), which has a constant thickness with two opposite surfaces of the same form as the surface 35, its diameter and thickness being the same as those of the surface 36.

When the filtration body 2, the drainage body 3 and the membrane 4 are assembled, as shown notably in FIGS. 1 and 2, the membrane 4 is gripped between the edge of the lateral wall 9 of the reservoir 5 of the intake body 2 and the surface 33 of the wall 32 of the circular table 30 of the drainage body 3. The bodies 2 and 3 are locked to one another by virtue of the latching tabs 7 and the skirt 31, which are mutually disposed as can be seen more particularly in FIG. 5.

The tooth 45 of the wall 42 fits into the groove 23 of the tab 7 and that the shoulder 24 of this tab fits into the space situated between the wall 43 and the tooth 45, so that the cooperation between the shoulder 24 and the tooth 45 provides an extremely powerful locking of the tab 7 in the skirt 31, capable of withstanding relatively large forces tending to move the bodies 2 and 3 away from one another.

The end 18 of the tab 7 is recessed with respect to the free end of the wall 43, so that, when the device 1 is put down on a surface with the drainage body 3 at the bottom, it is by means of the skirt 31 thereof that the device 1 rests on this surface, no force being exerted for this reason on the tabs 7, which therefore do not risk being broken accidentally.

As can be seen in FIG. 2, when the device 1 is assembled, the seal 13 and more particularly, the cushion thereof, is highly compressed compared with the off-load form of this seal shown in FIG. 6. The seal 13 has a T-shaped general profile whose longitudinal branch forms a rib 50 designed to be inserted into the groove 14 and whose transverse branch forms a cushion 51 designed to enter into contact with the membrane 4. The free end of the cushion 51 has a central slot 52 that makes it possible to release two annular lips 53 allowing the best cooperation of the cushion 51 with the membrane 4. The junction between the rib 50 and the cushion 51 is made by a straight surface on the internal side while, on the external side, t here is a bevel 54. The bevel 54 corresponds to a chamfered lip 55 at the external periphery of the end of the rigid part of the wall 9, this chamfered lip making it possible to laterally contain the cushion 51 on the external side in order that it flows mainly inward, that is to say towards the chamber delimited by the membrane 4 and the reservoir 5.

The intake body 2 is obtained, with the exception of the seal 13, by molding of a relatively rigid and transparent plastic, and then there is molded on to this piece, the seal 13, which is made of elastomer, this over-molding being carried out in the example illustrated by bi-injection. This part of the drainage body 3 depicted in FIG. 4 includes porous pad 48.

In order to assemble the device 1, the drainage body 3 and the membrane 4, are positioned concentrically. The intake body 2 is positioned facing the drainage body 3 with the latching tabs 7 aligned with the openings 44. The body 2 is pressed hard towards the body 3 so that the tabs 7 engage in the openings 44 flexing slightly by virtue of the inclined surface 21 that acts as a ramp. The force exerted allows the surface 22 of the shoulder 24 to ride over the tooth 45 at the end of the pushing in movement, by virtue of the spring of the tabs 7. The seal 13 relaxes so that the play between the tabs 7 and the skirt 31 is completely taken up. The elasticity of the seal 13 is then compressed, maintaining the locking thus obtained. By maintaining of the seal in the compressed state provides excellent sealing between the membrane 4 and the edge of the wall 9, and furthermore, by reaction, between the membrane 4 and the surface 33.

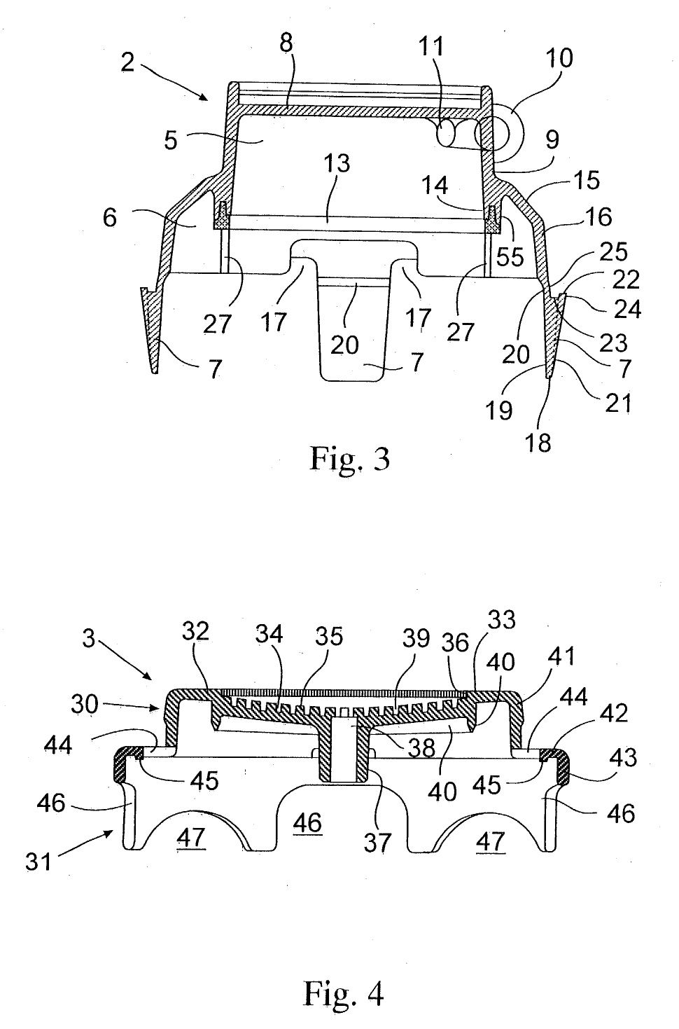

The internal surface of the wall 16 has localized areas of extra thickness 27 (FIG. 3) coming into contact with the external surface of the wall 41, which provides a lateral wedging between these surfaces, which are of similar diameter, and more generally between the bodies 2 and 3. Once the device 1 has been assembled in this way, it is possible to package it and sterilize it with a gas such as ethylene oxide or by irradiation. Prior to packaging the assembled device 1 and sterilizing it, each of the conduits 10 and 37 is equipped with a stopper.

When using the device 1, the stoppers blocking off the conduit 10 and the stopper 40a blocking conduit 37 are removed, then the conduit 10 is connected to a source of liquid under pressure, for example using, as shown in FIG. 7, a sampling connector 60 having a male luer tip 61. The tip 61 is inserted into the passage of the conduit 10 and the valve 62 of the connector 60 is manipulated, so that the chamber formed by the reservoir 5 and the membrane 4 is raised to a pressure intermediate between atmospheric pressure and the liquid pressure inside the apparatus (a few psi is the liquid is sampled from the bottom of a tank, to several bar if the liquid is sampled from pressurized pipes), for example 3 bars. The connector 60 is in fluid communication with conduit 60a such as a food processing conduit. The liquid entering the reservoir 5 by passing through the membrane 4 comes to rest on the porous pad 48. The liquid that has passed through the membrane 4 is guided by the channels 39 to the aperture 38. The liquid leaves the device 1 by the conduit 37. A graduated container 71 is preferably disposed under the device 1 in order to recover the liquid from conduit 37 in order to determine when the volume required for the sample has passed through the membrane 4.

When the desired measured volume has been reached, the valve 62 is closed and the device 1 is removed from the connector 60. A syringe or hand vacuum pump or other vacuum generating accessory, sealable under the device is then put in place under the drainage unit. The drainage of the liquid still present notably in the reservoir 5 is next carried out by suction through the output aperture 38.

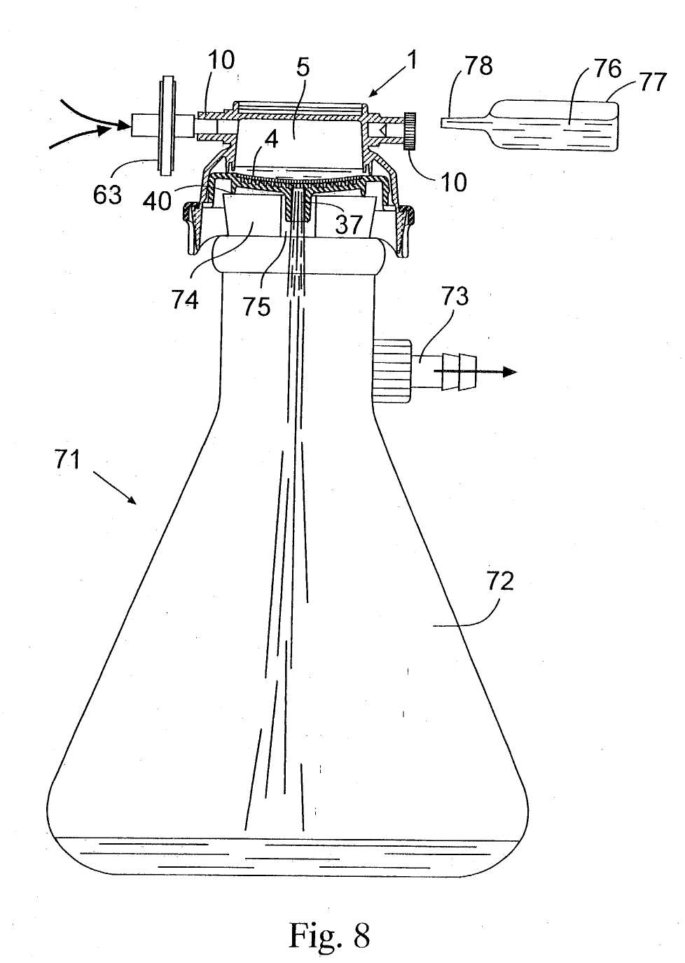

The notches 47 made in the wall 43 make it possible to correctly position the pump or syringe 64 in relation to the device 1, in four positions at 90° from one another, two of these positions being shown in FIG. 8.

The liquid remaining in the device 1 after sampling can also be removed with a vacuum flask, as shown in FIG. 8. The vacuum flask 71 illustrated has a glass body 72 having, at the level of its neck, a pipe 73 connected, in a manner not depicted, to a vacuum pump, and, at the top of this neck, a flexible stopper 74 with a central aperture 75 made in it, the flask 71 being of a type which is commonly found in practice. The device 1 is simply put down on the stopper 74, with the pipe 37 engaged in the aperture 75 and the rib 40 supported on the top of the stopper 74. On account of the tapered profile of the rib 40, the latter locally deforms the stopper 74 and provides sealing which makes it possible to suck out the residual liquid.

Once the liquid remaining in the device 1 has been emptied therefrom, it is removed from the flask 71 and the conduit 37 is stoppered with stopper 40a. A reagent 76 capable of lysing cells and of reacting with the lysed cell(s) as described above is enclosed in ampoule 77. The hollow tip 78 is broken and it is inserted into conduit 10. The ampoule 77 is preferably squeezed rather than relying on gravity to deliver the reagent 76 into container 5 where it resides to effect the desired reaction. The radiant energy emitted (luminescence) produced by the reaction then is measured through the transparent surface 8 such as with a luminometer.

Filters that may be used to retain the microbe cells are those commonly used in the industry for just such as purpose. Typically, any filter having a nominal pore size between 0.2 μm and 1.2 μm is useful in this invention. However, as one wishes to maintain an adequate flow rate, filters having a nominal pore size of about 0.45 micron are preferred. These may be made of various materials including but limited to mixed cellulose esters, regenerated cellulose, PVDF, PES, nylons, and the like. Such filters are commercially available from Millipore Corporation of Billerica, Mass. under the various brandnames such as Durapore® PVDF filters, S-Pak™HA, HC, AO and SO mixed cellulosic ester filters and Express® and Express®Plus PES asymmetric filters.

Additional useful parameters for the system include selecting materials for the device and filter that have a low fluorescence so as to minimize the level of background fluorescence or noise that might occur from the various components of the system and interfere with the reading of the lysed cells by for example, the luminometer.

As mentioned above, adding reagents to eliminate non-microbial cells and/or their components such as ATP.DNA, etc. that would compete with reagent is also useful. See for example U.S. Pat. No. 6,465,201 in which mammalian cells are selected lysed, treated with enzymes to degrade or eliminate the ATP within those cells, such as ATP hydrolyzing enzymes, including but not limited to adenosine triphosphatase and then subjecting the filter to a washing step before lysing the microbial cells is one such method that is useful in this invention when non-microbial cell contamination is known or suspected leading to a truer result.

Minimizing the distance between the filter and the illumination sensor is preferred as it maximizes the optical detection of the system. Likewise, having the filtration area being of the same or similar surface area and configuration of the illumination sensor is preferred so it reduces or eliminates the potential for any lost signal by a microbe that is on an area of the filter but not within the detection area of the sensor.

What is claimed:

1. A kit, for use on-site, to rapidly determine microbe concentration in a liquid sample in conjunction with a radiant energy detector that comprises:
(a) an apparatus suitable for continuous filtration of large sample volume under pressure comprising a housing having an inlet, an outlet, means for supporting a membrane filter positioned between said inlet and said outlet, a filter mounted on the means for supporting the filter for retaining microbes, a transparent top surface and means for sealing said housing from a surrounding atmosphere, and
(b) at least one container for housing a reagent composition including a first reactant for lysing microbe cells and a second reactant for reacting with lysed cells to produce radiant energy,
(c) a portable apparatus to detect and measure the radiant energy, including the measurement of time from which the filter apparatus is exposed to the reagents, the total reaction time, as well as a means to express the radiant energy compared to pre-set acceptable values, and protecting the filtration device from reading contamination by environmental radiation during reading.

2. The kit of claim 1 which includes two containers for housing said first reactant and said second reactant.

3. The kit of claim 1 wherein said filter is supported on a porous pad.

4. The kit of claim 1 wherein said filter is supported on a porous pad and said housing includes drainage channels under said porous pad, said drainage channels opening into said outlet.

5. The kit of claim 1 wherein said first reactant is selected from the group consisting of detergents, alcohols, esters, ethers, halogenated derivatives of methane, ethane, methylene and ethylene, acetonitrile, trimethylamine and mixtures thereof and said second reactant is selected from the group consisting of luciferine and luciferase, radioactive tags and one or more PNA tags.

6. The kit of claim 1 wherein said first reactant is selected from the group consisting of detergents, methanol and ethanol and said second reactant is luciferine and luciferase.

7. The kit of claim 1 wherein said first reactant is selected from the group consisting of detergents, methanol and ethanol and said second reactant is one or more PNA tags.

8. The process for determining microbe concentration in a liquid sample which comprises:
passing a liquid sample through an apparatus comprising housing having an inlet, an outlet, means for supporting a membrane filter positioned between said inlet and said outlet, a membrane filter mounted to said means fro supporting a membrane filter, a transparent top surface and means for sealing said housing from surrounding atmosphere and to isolate microbes in said liquid sample on said membrane filter,
introducing a reagent composition comprising a first reactant for lysing microbe cells and a second reactant for reacting with lysed cells to produce radiant energy,
sealing said housing containing first reactant and second reactant, said atmosphere, and measuring radiant energy from said housing.

9. The process of claim 8 wherein said first reactant is selected from the group consisting of detergents, alcohols, esters, ethers, halogenated derivatives of methane, ethane, methylene and ethylene; acetonitrile, trimethylamine and mixtures thereof and said second reactant is selected from the group consisting of luciferine and luciferase, radioactive tags and one or more PNA tags.

10. The device of claim 1 wherein the filter is of about the same size and configuration as the active detection surface of the sensor.

11. The process of claim 8 further comprising eliminating any cells other than the microbe cells before lysing the microbe cells.

12. The process of claim 8 further comprising eliminating any cells other than the microbe cells before lysing the microbe cells by selectively lysing all cells other than the microbe cells, degrading their contents by enzymes and washing the filter to remove the degraded contents.

13. The device of claim 1 wherein the apparatus has a low level of fluorescence.

14. The device of claim 1 wherein the apparatus has a low level of material that is capable of reacting with the second reagent.
